(19) United States
(12) Patent Application Publication
(10) Pub. No.: US 2006/0121087 A1
(43) Pub. Date: Jun. 8, 2006

(54) Polymeric endoprostheses with modified erosion rates and methods of manufacture

(76) Inventors: Michael S. Williams, Santa Rosa, CA (US); Joseph M. DeSimone, Chapel Hill, NC (US)

Correspondence Address: DEANNA J. SHIRLEY, 3418 BALDWIN WAY, SANTA ROSA, CA, 95403, US

(21) Appl. No.: 11/274,520

(22) Filed: Nov. 15, 2005

Related U.S. Application Data
(60) Provisional application No. 60/633,494, filed on Dec. 6, 2004.

(51) Int. Cl. A61F 2/00

(52) U.S. Cl. 424/426

(57) ABSTRACT

An erodible prosthesis comprising alternate rates of erosion is disclosed, wherein said alternate rates of erosion can be selectively initiated. Some embodiments according to the invention may comprise an agent for initiating an alternative rate of erosion, such as, for example, a sensitizer, dissolution inhibitor, photo-acid generator, biochemically active additive, thermally activated catalyst, light activated catalyst, electromagnetic radiation activated catalyst, hydration activated catalyst, pH activated catalyst, low melting agent, and/or enzyme activated catalyst. One or more of the foregoing agents may be dispersed within one or more layers.

### RELATED APPLICATIONS

This application is related to and claims the benefit of the priority date of U.S. Provisional Patent Application Ser. No. 60/633,494 entitled “Polymeric Endoprostheses with Modified Erosion Rates and Methods of Manufacture”, filed Dec. 6, 2004.

### FIELD OF THE INVENTION

The invention herein relates generally to medical devices and the manufacture thereof, and to improved endoprostheses and methods for manufacturing endoprostheses. Endoprostheses disclosed herein may be for use in the treatment of strictures in lumens of the body, devices used to occlude a lumen, in the treatment of other cardiovascular disorders, treatment of gastrointestinal disorders, ocular disease, degenerative diseases of the spine, degeneration and/or trauma to bone or muscle, or may be implanted to treat other disorders. More particularly, the inventions disclosed herein are directed to erodible polymeric endoprostheses and address the shortcomings of the prior art by providing, for example, controlled and cycled rates of erosion.

### BACKGROUND OF THE INVENTION

Implantable medical devices that may be permanent or erodible have revolutionized treatment of many disorders, including but not limited to coronary artery disease, biliary, esophageal, and gastrointestinal disorders, ureteral dysfunction, disorders of the eye, disorders of the spine, and degeneration and trauma to bone. Many such devices may be implanted via minimally invasive techniques, thereby reducing hospitalization and recovery time for patients. Successful treatment may require continued monitoring of a significant portion of the relevant patient population. Magnetic resonance imaging (MRI) is currently emerging as the state of the art diagnostic, enhancing the detection, diagnosis and monitoring of many disorders. Polymeric endoprostheses, which do not cause distortion of MRI images, are readily compatible with MRI.

Continued improvements in implantable medical device technology aim at producing easily tracked, easily visualized and readily deployed devices comprising the requisite mechanical properties for treating a given disorder. In addition, in situ drug delivery, gene therapy and other therapies can be successfully coupled with implanted mechanical devices. Many such devices ideally exhibit particular mechanical and/or chemical properties for a desired period of time, and one or more alternative sets of properties for another period of time, and perhaps alternating between sets of desired properties. Such a device may then erode entirely or remain indefinitely, and may exhibit desired mechanical properties for the remainder of the life of the device.

While advances have been made in the use of implantable devices or endoprostheses to treat many disorders, there remains a need for devices that erode at desired rates and/or with relatively controlled cycles of erosion and/or drug delivery.

### SUMMARY OF THE INVENTION

An erodible polymeric endoprosthesis comprising a first rate of erosion and a second rate of erosion is disclosed, wherein the first rate of erosion may exist during a first period of time and the second rate of erosion exists during a second period of time, or wherein the first rate of erosion and second rate of erosion occur simultaneously. An endoprosthesis according to the invention may further comprise additional alternative rates of erosion. The erodible polymeric endoprosthesis may comprise a first set of mechanical properties during a first period of time that can vary as a function of time and a second set of mechanical properties during a second period of time that can vary as a function of time. The endoprosthesis may comprise a therapeutic substance, wherein the therapeutic substance is released from said endoprosthesis at an increased or decreased rate during the first period of time or during the second period of time.

Some embodiments according to the invention may comprise an agent for initiating or terminating erosion or initiating an alternate rate of erosion. The agent may be selected from the group consisting of: sensitizers, dissolution inhibitors, photo-acid generators, biochemically active additives, thermally activated catalysts, light activated catalysts, electromagnetic radiation activated catalysts, hydration activated catalysts, pH activated catalysts, low melting agents, and enzyme activated catalysts.

An endoprosthesis according to the invention may further comprise a first layer and a second layer or more layers, wherein the first layer comprises a first rate of erosion and said second layer comprises a second rate of erosion. The first layer may comprises a polymer resin. A layer exhibiting a relatively slower rate of erosion may comprise a polymer comprising a protective group. One or more layers may further comprise a photo-acid generator, a dissolution inhibitor, a low-melting agent, or other agent for initiating a change in erosion rate.

A change in rate of erosion may be initiated by the exposure of the endoprosthesis to one or more stimuli. The stimulus may selected from the group consisting of: change in temperature, change in pH, light, electromagnetic radiation, hydration, one or more biochemical catalysts, and one or more enzymes. A change in rate of erosion may result from the removal of a protective group from the resin.

A method of manufacture of an endoprosthesis comprising one or more alternate rates of erosion is disclosed, the method comprising the steps of providing a polymer resin comprising a relatively high rate of erosion; reacting the polymer with a functional group, thereby decreasing the polymer's rate of erosion; embedding an agent for selectively increasing or decreasing the polymer's rate of erosion in the polymer; and fabricating an endoprosthesis from the polymer.

The agent may be selected from the group consisting of: sensitizers, dissolution inhibitors, photo-acid generators, biochemically active additives, thermally activated catalysts, light activated catalysts, electromagnetic radiation activated catalysts, hydration activated catalysts, pH activated catalysts, low melting agents, and enzyme activated catalysts.

A method of manufacture may include the additional step of introducing a catalyst that initiates a reaction or a series of reactions that result in an increased or decreased rate of erosion. The reaction or series of reactions may result in a decreased molecular weight of the polymer and/or deprotection of a functional group.

An alternative method of manufacture of an endoprosthesis comprising one or more alternate rates of erosion may comprise the steps of: providing a polymer comprising a relatively low rate of erosion; embedding an agent for selectively increasing or decreasing the polymer's rate of erosion in the polymer; and fabricating an endoprosthesis from the polymer. The method may comprise the additional step of introducing a catalyst that initiates a reaction or a series of reactions that result in an increased or decreased rate of erosion, and/or a decreased molecular weight of the polymer.

A method of manufacture of an endoprosthesis comprising one or more alternate sets of mechanical properties comprises the steps of providing a polymer comprising a relatively low rate of erosion; embedding an agent for selectively increasing or decreasing the polymer's rate of erosion in the polymer, and fabricating an endoprosthesis from the polymer. In any of the foregoing methods of manufacture, the method may comprise the additional step of incorporating a therapeutic substance into the endoprosthesis.

A method of treatment of a subject is disclosed, comprising the steps of providing an erodible endoprosthesis comprising one or more alternate rates of erosion; implanting the endoprosthesis in the subject; and selectively initiating an increased or decreased rate of erosion of the endoprosthesis. The increased or decreased rate of erosion may continue for a relatively predetermined period of time, and may be followed by a relatively slower or higher rate of erosion, or continue until the endoprosthesis is substantially completely eroded. A stimulus for initiation of a reaction or a series of reactions that result in an increased or decreased rate of erosion may be introduced.

The endoprosthesis may comprise an agent for selectively increasing or decreasing the rate of erosion of the endoprosthesis, and the agent may be responsive to the introduction of a stimulus.

### BRIEF DESCRIPTION OF THE DRAWINGS

FIG. 1 is a flow chart illustrating a first embodiment according to the invention.

FIG. 2 is a second flow chart illustrating an alternative embodiment according to the invention.

FIG. 3 is a third flow chart illustrating yet another embodiment according to the invention.

### DETAILED DESCRIPTION OF THE INVENTION

Although the invention herein is not limited as such, some embodiments of the invention comprise materials that are bioerodible. “Erodible” refers to the ability of a material to maintain its structural integrity for a desired period of time, and thereafter gradually undergo any of numerous processes whereby the material substantially loses tensile strength and mass. Examples of such processes comprise hydrolysis, enzymatic and non-enzymatic degradation, oxidation, enzymatically-assisted oxidation, and others, thus including bioresorption, dissolution, and mechanical degradation upon interaction with a physiological environment into components that the patient's tissue can absorb, metabolize, respire, and/or excrete. Polymer chains are cleaved by hydrolysis and are eliminated from the body through the Krebs cycle, primarily as carbon dioxide and in urine. “Erodible” and “degradable” are intended to be used interchangeably herein.

“Embedded” agents are set upon and/or within a mass of material by any suitable means including, but not limited to, combining the agent with the material while the material (such as, for example, a polymer) is in solution, combining the agent with the material when the material is heated near or above its melting temperature, affixing the agent to the surface of the material, and others. Examples of methods of embedding agents utilizing a solvent in a supercritical state are set forth in U.S. patent application Ser. Nos. 10/662,757 and 10/662,621, and are incorporated as if fully set forth herein.

A “self-expanding” endoprosthesis has the ability to revert readily from a reduced profile configuration to a larger profile configuration in the absence of a restraint upon the device that maintains the device in the reduced profile configuration.

“Balloon expandable” refers to a device that comprises a reduced profile configuration and an expanded profile configuration, and undergoes a transition from the reduced configuration to the expanded configuration via the outward radial force of a balloon expanded by any suitable inflation medium.

The term “balloon assisted” refers to a self-expanding device the final deployment of which is facilitated by an expanded balloon.

The term “fiber” refers to any generally elongate member fabricated from any suitable material, whether polymeric, metal or metal alloy, natural or synthetic.

The phrase “points of intersection”, when used in relation to fiber(s), refers to any point at which a portion of a fiber or two or more fibers cross, overlap, wrap, pass tangentially, pass through one another, or come near to or in actual contact with one another.

As used herein, a device is “implanted” if it is placed within the body to remain for any length of time following the conclusion of the procedure to place the device within the body.

The term “diffusion coefficient” refers to the rate by which a substance elutes, or is released either passively or actively from a substrate.

As used herein, the term “braid” refers to any braid or mesh or similar woven structure produced from between 1 and several hundred longitudinal and/or transverse elongate elements woven, braided, knitted, helically wound, or intertwined by any manner, at angles between 0 and 180 degrees and usually between 45 and 105 degrees, depending upon the overall geometry and dimensions desired.

Unless specified, suitable means of attachment may include by thermal melt, chemical bond, adhesive, sintering, welding, or any means known in the art.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric. A polymer is a shape memory polymer if the original shape of the polymer is substantially recovered by heating it above a shape recovering temperature (defined as the transition temperature of a soft segment) even if the original molded shape of the polymer is destroyed mechanically at a lower temperature than the shape recovering temperature, or if the memorized shape is recoverable by application of another stimulus. Such other stimulus may include but is not limited to pH, salinity, hydration, and others.

As used herein, the term “segment” refers to a block or sequence of polymer forming part of the shape memory polymer. The terms hard segment and soft segment are relative terms, relating to the transition temperature of the segments. Generally speaking, hard segments have a higher glass transition temperature than soft segments, but there are exceptions. Natural polymer segments or polymers include but are not limited to proteins such as casein, gelatin, gluten, zein, modified zein, serum albumin, and collagen, and polysaccharides such as alginate, chitin, celluloses, dextrans, pullulane, and polyhyaluronic acid; poly(3-hydroxyalkanoate)s, especially poly(.beta.-hydroxybutyrate), poly(3-hydroxyoctanoate) and poly(3-hydroxyfatty acids).

Representative natural erodible polymer segments or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Suitable synthetic polymer blocks include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, arboxymethyl cellulose, cellulose triacetate and cellulose sulfate sodium salt. These are collectively referred to herein as “celluloses”.

Examples of synthetic degradable polymer segments or polymers include polyhydroxy acids, polylactides, polyglycolides and copolymers thereof, poly(ethylene terephthalate), poly(hydroxybutyric acid), poly(hydroxyvaleric acid), poly[lactide-co-(epsilon-caprolactone)], poly[glycolide-co-(epsilon-caprolactone)], polycarbonates, poly-(epsilon caprolactone) poly(pseudo amino acids), poly(amino acids), poly(hydroxyalkanoate)s, polyanhydrides, polyortho esters, and blends and copolymers thereof.

The degree of crystallinity of the polymer or polymeric block(s) is between 3 and 80%, more often between 3 and 65%. The tensile modulus of the polymers below the transition temperature is typically between 50 MPa and 2 GPa (gigapascals), whereas the tensile modulus of the polymers above the transition temperature is typically between 1 and 500 MPa.

The melting point and glass transition temperature (T) of the hard segment are generally at least 10 degrees C., and preferably 20 degrees C., higher than the transition temperature of the soft segment. The transition temperature of the hard segment is preferably between −60 and 270 degrees C., and more often between 30 and 150 degrees C. The ratio by weight of the hard segment to soft segments is between about 5:95 and 95:5, and most often between 20:80 and 80:20. The polymers contain at least one physical crosslink (physical interaction of the hard segment) or contain covalent crosslinks instead of a hard segment. Polymers can also be interpenetrating networks or semi-interpenetrating networks.

Rapidly erodible polymers such as poly(lactide-co-glycolide)s, polyanhydrides, and polyorthoesters, which have carboxyl groups exposed on the external surface as the smooth surface of the polymer erodes, also can be used. In addition, polymers containing labile bonds, such as polyanhydrides and polyesters, are well known for their hydrolytic reactivity. Their hydrolytic degradation rates can generally be altered by simple changes in the polymer backbone and their sequence structure.

Examples of suitable hydrophilic polymers include but are not limited to poly(ethylene oxide), polyvinyl pyrrolidone, polyvinyl alcohol, poly(ethylene glycol), polyacrylamide poly(hydroxy alkyl methacrylates), poly(hydroxy ethyl methacrylate), hydrophilic polyurethanes, HYPAN, oriented HYPAN, poly(hydroxy ethyl acrylate), hydroxy ethyl cellulose, hydroxy propyl cellulose, methoxylated pectin gels, agar, starches, modified starches, alginates, hydroxy ethyl carbohydrates and mixtures and copolymers thereof.

Hydrogels can be formed from polyethylene glycol, polyethylene oxide, polyvinyl alcohol, polyvinyl pyrrolidone, polyacrylates, poly (ethylene terephthalate), poly(vinyl acetate), and copolymers and blends thereof. Several polymeric segments, for example, acrylic acid, are elastomeric only when the polymer is hydrated and hydrogels are formed. Other polymeric segments, for example, methacrylic acid, are crystalline and capable of melting even when the polymers are not hydrated. Either type of polymeric block can be used, depending on the desired application and conditions of use.

The use of polymeric materials in the fabrication of endoprostheses confers the advantages of improved flexibility, compliance and conformability, and controlled rate of erosion, permitting treatment in body lumens not accessible by more conventional endoprostheses.

Fabrication of an endoprosthesis according to the invention allows for the use of different materials in different regions of the prosthesis to achieve different physical properties as desired for a selected region. A material selected for its ability to allow elongation of longitudinal connecting members on the outer radius of a curve in a lumen, and compression on the inner radius of a curve in a vessel allows improved tracking of a device through a diseased lumen. A distinct material may be selected for support elements in order that the support elements exhibit sufficient radial strength. Further, the use of polymeric materials readily allows for the fabrication of endoprostheses comprising transitional end portions with greater compliance than the remainder of the prosthesis, thereby minimizing any compliance mismatch between the endoprosthesis and diseased lumen. Further, a polymeric material can uniformly be processed to fabricate a device exhibiting better overall compliance with a pulsating vessel, which, especially when diseased, typically has irregular and often rigid morphology. Trauma to the vasculature, for example, is thereby minimized, reducing the incidence of restenosis that commonly results from vessel trauma.

An additional advantage of polymers includes the ability to control and modify properties of the polymers through the use of a variety of techniques. According to the invention, optimal ratios of combined polymers, optimal configuration of polymers synthesized to exhibit predictable rates of erosion, and optimal processing have been found to achieve highly desired properties not typically found in polymers. In general, erosion of a polymer will progress at a known range of rates. Environmental factors such as pH, temperature, tissue or blood interaction and other factors such as structural design of the device all impact the degradation rate of erodible polymers. Depending upon the desired performance characteristics of a device, in some cases it may be desirable to either “program in” a desired rate of erosion, or desired cycle of varied rates of erosion, to initiate on-demand erosion of a device, or to have a set of desired mechanical properties or to function in a desired manner for a period of time, and an alternative set of desired mechanical properties for a second period of time. For example, it may be desirable for the device to deliver a therapeutic substance under particular conditions and/or during a particular time period.

According to the invention, a polymer may be tailored to erode rapidly during one phase, such as, for example, a drug delivery phase, followed by a period of time during which the polymer erodes at a slower rate. Such a time period of slower erosion may be followed by a second drug delivery phase during which the polymer again erodes rapidly. Similarly, a polymer may be tailored to erode on demand, upon the introduction of a stimulus such as increase in temperature, exposure to radiation, and/or others. Any number of combinations of desired phases is possible according to the invention.

The rate of erosion of a polymer may be controlled by one or more of several techniques. An example of such a technique includes the incorporation of an agent or substance that acts as a catalyst of degradation upon exposure to a stimulus. Examples of such agents or substances include, but are not limited to, sensitizers, dissolution inhibitors, biochemically active additives, thermal, light, electromagnetic radiation, or enzyme-activated catalysts, or some combination of the foregoing. Examples of sensitizers include, but are not limited to photoacid generators (PAGs), dissolution inhibitors, and radiosensitizers. Examples of biochemically active additives include, but are not limited to, lipids. Further, one or more layers of polymer comprising one of the foregoing agents may alternate with a layer of polymer that does not comprise such an agent, or is tailored to erode at a different rate or upon the introduction of an alternate stimulus.

According to another aspect of the invention, surface treatment and/or incorporation of therapeutic substances may be performed utilizing one or more of numerous processes that utilize carbon dioxide fluid, e.g., carbon dioxide in a liquid or supercritical state. A supercritical fluid is a substance above its critical temperature and critical pressure (or “critical point”). Compressing a gas normally causes a phase separation and the appearance of a separate liquid phase. However, all gases have a critical temperature above which the gas cannot be liquefied by increasing pressure, and a critical pressure or pressure which is necessary to liquefy the gas at the critical temperature. For example, carbon dioxide in its supercritical state exists as a form of matter in which its liquid and gaseous states are indistinguishable from one another. For carbon dioxide, the critical temperature is about 31 degrees C (88 degrees D) and the critical pressure is about 73 atmospheres or about 1070 psi.

The term “supercritical carbon dioxide” as used herein refers to carbon dioxide at a temperature greater than about 31 degrees C and a pressure greater than about 1070 psi. Liquid carbon dioxide may be obtained at temperatures of from about −15 degrees C to about −55 degrees C and pressures of from about 77 psi to about 335 psi. One or more solvents and blends thereof may optionally be included in the carbon dioxide. Illustrative solvents include, but are not limited to, tetrafluoroisopropanol, chloroform, tetrahydrofuran, cyclohexane, and methylene chloride. Such solvents are typically included in an amount, by weight, of up to about 20%.

In general, carbon dioxide may be used to effectively lower the glass transition temperature of a polymeric material to facilitate the infusion of pharmacological agent(s) into the polymeric material. Such agents include but are not limited to hydrophobic agents, hydrophilic agents and agents in particulate form. For example, following fabrication, an endoprosthesis and a hydrophobic pharmacological agent may be immersed in supercritical carbon dioxide. The supercritical carbon dioxide “plasticizes” the polymeric material, that is, it allows the polymeric material to soften at a lower temperature, and facilitates the infusion of the pharmacological agent into the polymeric endoprosthesis or polymeric coating of a stent at a temperature that is less likely to alter and/or damage the pharmacological agent.

As an additional example, an endoprosthesis and a hydrophilic pharmacological agent can be immersed in water with an overlying carbon dioxide “blanket”. The hydrophilic pharmacological agent enters solution in the water, and the carbon dioxide “plasticizes” the polymeric material, as described above, and thereby facilitates the infusion of the pharmacological agent into a polymeric endoprosthesis or a polymeric coating of an endoprosthesis.

As yet another example, carbon dioxide may be used to “tackify”, or render more fluent and adherent a polymeric endoprosthesis or a polymeric coating on an endoprosthesis to facilitate the application of a pharmacological agent thereto in a dry, micronized form. A membrane- forming polymer, selected for its ability to allow the diffusion of the pharmacological agent therethrough, may then applied in a layer over the endoprosthesis. Following curing by suitable means, a membrane that permits diffusion of the pharmacological agent over a predetermined time period forms.

Objectives of therapeutics substances incorporated into materials forming or coating an endoprosthesis according to the invention include reducing the adhesion and aggregation of platelets at the site of arterial injury, block the expression of growth factors and their receptors; develop competitive antagonists of growth factors, interfere with the receptor signaling in the responsive cell, promote an inhibitor of smooth muscle proliferation. Anitplatelets, anticoagulants, antineoplastics, antifibrins, enzymes and enzyme inhibitors, antimitotics, antimetabolites, anti-inflammatories, antithrombins, antiproliferatives, antibiotics, anti-angiogenesis factors, and others may be suitable.

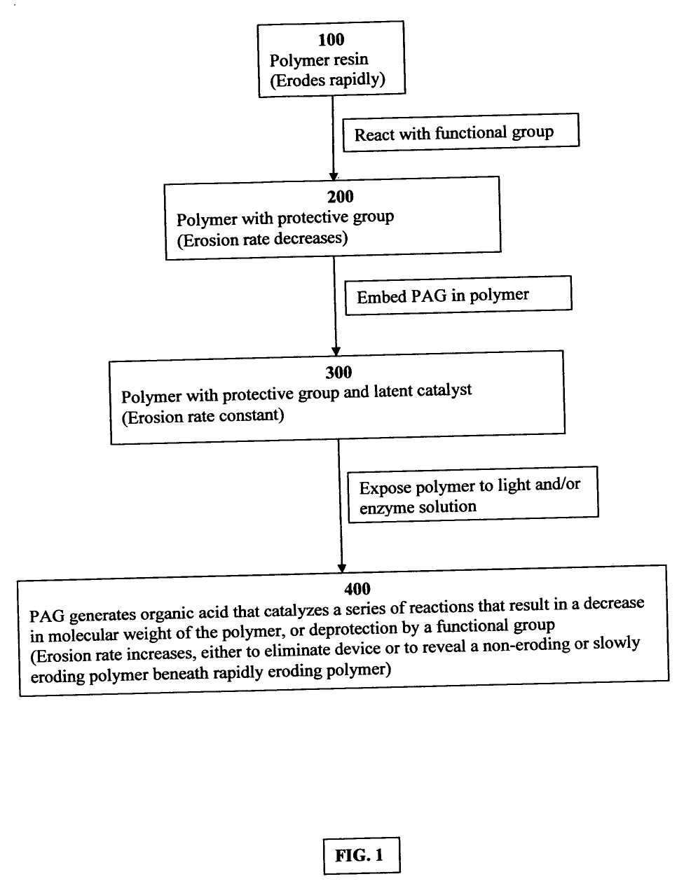

Details of the invention can be better understood from the following descriptions of specific embodiments according to the invention. As an example illustrated in FIG. 1, an implantable device may comprise a polymer resin which is very soluble in aqueous media due to the presence of hydroxyl groups (100). In order to synthesize a less soluble polymer, these hydroxyl groups may be “blocked” by reacting the hydroxyl group with a molecule, such as a tert-butoxycarbonyl (t-BOC group), a comparable functional group, or an alkyl ester. The polymer, in this form, and consequently the device, will erode very slowly (200).

According to the invention, in order to design the polymer that will, upon demand, erode more rapidly, the polymer may additionally comprise a photoacid generator (PAG) such as dinitrobenzyl tosylate embedded therein. (300). Upon exposure to light, a photoacid generator degrades to generate an organic acid locally. The organic acid may act as a catalyst of a series of reactions that lower the molecular weight of the polymer, consequently rendering the polymer more susceptible to degradation, and thereby increasing the rate of degradation of the polymer (400). Alternatively, the acid generated by the PAG may trigger the deprotection of a functional group, such as a t-BOC group, which would significantly increase the rate of solubility and/or swellability of the polymer in hydrophilic media (400).

Following deployment of a device comprising a polymer manufactured according to the invention, the device erodes very slowly. Also according to the invention, a clinician may then, or at some later time, initiate degradation of a portion of or the entire device. Such on-demand degradation may be commenced by the controlled local delivery of light to the device, via, for example, a minimally invasive catheterization. The light exposure initiates the degradation of the PAG, thereby setting in motion the sequence of events set forth above to erode the polymer. Alternatively, or in addition, an enzymatic solution may be delivered via catheter in order to initiate a series of reactions that lead to an increased or decreased rate of degradation of the polymer and device.

An alternative to the example of FIG. 1 is a polymeric implantable device that comprises a dissolution inhibitor such as, for example, diazonapthaquinone. Such a dissolution inhibitor, similar to the example set forth above, may be synthesized to comprise protective groups that dramatically decrease the solubility of the polymer. Upon exposure to light or other stimulus that may trigger the deprotection of a functional group, a dissolution inhibitor then dramatically enhances the rate of dissolution of a polymer. Similar to the example set forth above, a clinician may deliver light or an enzymatic solution locally in order to initiate an increased or decreased rate of erosion of the device. Such a polymeric device may further comprise a therapeutic agent that is consequently released from the polymer upon degradation of the device. Regardless of the mechanism and/or catalyst of erosion, either the entire device may be eliminated or merely a first layer of the device may be eroded to reveal a non-eroding or slowly eroding material beneath the substantially completely eroded outermost layer.

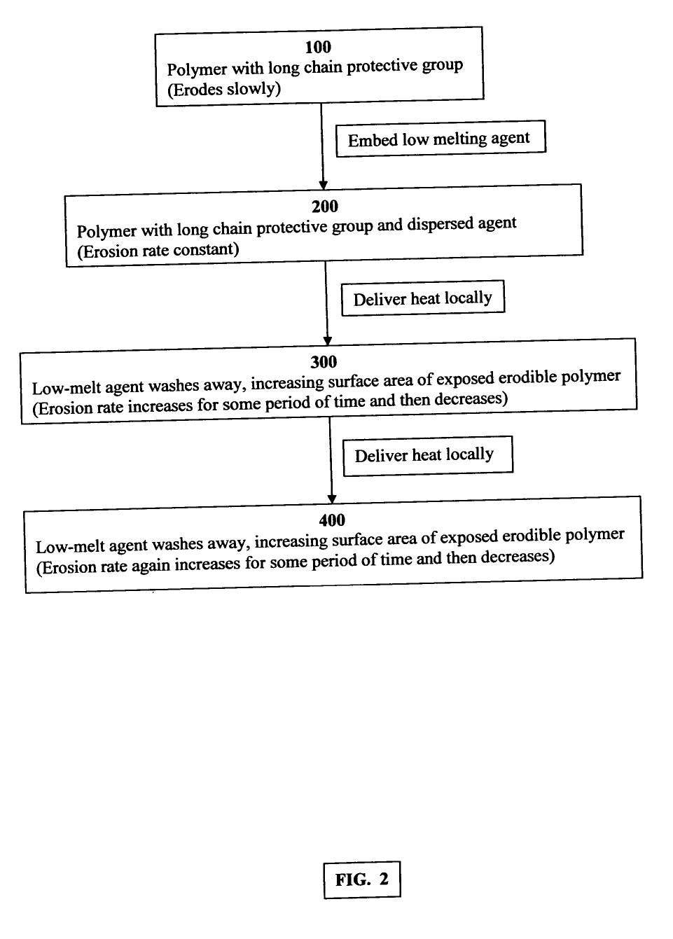

Turning now to FIG. 2, additional examples of techniques for initiating polymer degradation are illustrated. Polymer degradation may be initiated thermally. For example, a t-BOC blocked polymer undergoes acidolysis to generate the soluble hydroxyl group in the presence of acid (as described above) and heat. Further, a polymer may be synthesized to comprise a latent catalyst that, upon exposure to heat, greatly increases the degradation of the device. Further yet, heat may initiate either a phase change or a morphological change which triggers the degradation of the device, such as, for example, a melting transition.

For example, as illustrated in FIG. 2, the device may comprise a low melting salt or wax embedded throughout the polymer that liquefies upon thermal treatment and is washed/dissolved away rapidly. The removal of the low melting agent from the exposed surface area of the device increases the exposed surface area of the polymer, thereby facilitating an increased or decreased rate of degradation of the polymer. Safe and effective local delivery of heat may be achieved via minimally invasive techniques, such as a CT scan, or MRI-based “real time” control. Repeated cycles of heat delivery, separated by desired time intervals of, for example, weeks, months or even years, result in controlled cycles of polymer and device erosion rates. In the alternative to, or in combination with the foregoing, one or more desired agents may be variably dispersed within alternate layers of polymer. Such a configuration may achieve, for example, rapid delivery of a first therapeutic agent, followed by a sustained delivery of the same or a second therapeutic agent, or some combination thereof. Similar to the example of FIG. 1, the foregoing device may be designed to erode in its entirety, or to reveal a subsequent layer of polymer designed to erode at a different rate or upon the exposure to an alternate catalyst.

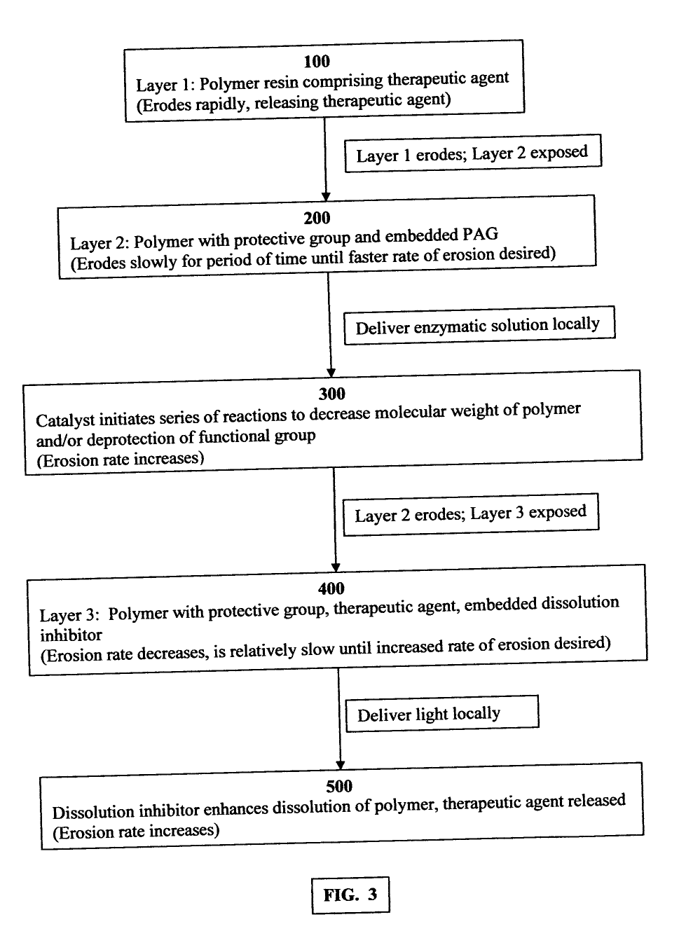

FIG. 3 illustrates an example of a combination of layers of polymers comprising varied rates of erosion. Other combinations may be desirable. The example of a device shown in FIG. 3 comprises a polymer comprising an outermost layer, “Layer 1” (100). Layer 1 comprises a relatively rapidly eroding polymer resin with a therapeutic agent. Layer 1 erodes rapidly, simultaneously delivering the therapeutic agent and eventually exposing “Layer 2”.

Layer 2 comprises a more slowly eroding polymer in which a PAG is embedded (200). Layer 2 erodes relatively slowly for a period of time. When, under particular circumstances, a more rapid rate of erosion is desired, the clinician may deliver an enzyme solution locally, which through a series of reactions and via several mechanisms, initiates an increased rate of erosion of layer 2 (300). Layer 2 then erodes to expose “Layer 3”.

Layer 3 comprises a polymer comprising a long chain protective group, a therapeutic agent, and a dissolution inhibitor (400). In the absence of a catalyst, Layer 3 erodes at a relatively slow rate. When an increased rate of erosion is desired, the clinician may deliver light locally as described above. Light “converts” the dissolution inhibitor to enhance dissolution of the polymer, thereby increasing the rate of erosion of the polymer and the delivery of the therapeutic agent.

One or more layers may alternatively comprise lipids, which degrade in the presence of lipase, an enzyme found in blood. Erosion of lipids that are dispersed within a polymer increases the exposed surface area of degradable polymer, thereby increasing the rate of erosion.

As an additional alternative, one or more layers may comprise radiosensitizers, for example, O₂ endgroups. A radiosensitizer will degrade upon exposure to locally delivered radiation, thereby initiating an increased rate of erosion. Radiation may be delivered safely using minimally invasive techniques known in the art.

Alternative combinations of layers to those set forth above may be suitable. Further, other materials, agents and catalysts, both latent and active, may be substituted for those listed above according to the invention. In addition, the foregoing technology may be incorporated into any implant, including, without limitation, devices for use in the treatment of strictures in lumens of the body, devices used to occlude a lumen, in the treatment of other cardiovascular disorders, treatment of gastrointestinal disorders, ocular disease, degenerative diseases of the spine, degeneration and/or trauma to bone or muscle, or may be implanted to treat other disorders.

While particular forms of the invention have been illustrated and described above, the foregoing descriptions are intended as examples, and to one skilled in the art will it will be apparent that various modifications can be made without departing from the spirit and scope of the invention.

We claim:

1. An erodible polymeric endoprosthesis comprising a first rate of erosion and a second rate of erosion.

2. The endoprosthesis according to claim 1 wherein said endoprosthesis comprises said first rate of erosion during a first period of time and said second rate of erosion during a second period of time.

3. The endoprosthesis according to claim 2 wherein said endoprosthesis comprises a therapeutic substance, wherein said therapeutic substance is released from said endoprosthesis at an increased or decreased rate during said first period of time or during said second period of time.

4. The endoprosthesis according to claim 1, said endoprosthesis further comprising an agent for initiating said first rate of erosion or said second rate of erosion.

5. The endoprosthesis according to claim 4, wherein said agent is selected from the group consisting of: sensitizers, dissolution inhibitors, photo-acid generators, biochemically active additives, thermally activated catalysts, light activated catalysts, electromagnetic radiation activated catalysts, hydration activated catalysts, pH activated catalysts, low melting agents, and enzyme activated catalysts.

6. An erodible polymeric endoprosthesis comprising a first set of mechanical properties during a first period of time and a second set of mechanical properties during a second period of time.

7. The endoprosthesis according to claim 1 further comprising a first layer and a second layer, wherein said first layer comprises a first rate of erosion and said second layer comprises a second rate of erosion.

8. The endoprosthesis according to claim 7, wherein said first layer comprises a polymer resin.

9. The endoprosthesis according to claim 7, wherein said second layer comprises a polymer comprising a protective group.

10. The endoprosthesis according to claim 9 wherein said second layer further comprises a photo-acid generator.

11. The endoprosthesis according to claim 9, wherein said second layer further comprises a dissolution inhibitor.

12. The endoprosthesis according to claim 9, wherein said second layer further comprises a low-melting agent.

13. The endoprosthesis according to claim 1 wherein said second rate of erosion is selectively initiated by the exposure of said endoprosthesis to one or more stimuli.

14. The endoprosthesis according to claim 13 wherein said one or more stimuli is selected from the group consisting of: change in temperature, change in pH, light, electromagnetic radiation, hydration, one or more biochemical catalysts, and one or more enzymes.

15. The endoprosthesis according to claim 1, said endoprosthesis comprising a polymer comprising a protective group, wherein a reaction or a series of reactions results in removal of said protective group initiates said second rate of erosion.

16. A method of manufacture of an endoprosthesis comprising one or more alternate rates of erosion, said method comprising the steps:
providing a polymer resin comprising a relatively high rate of erosion;
reacting the polymer with a functional group, thereby decreasing the polymer's rate of erosion;
embedding an agent for selectively increasing or decreasing the polymer's rate of erosion in the polymer;
fabricating an endoprosthesis from the polymer.

17. The method according to claim 16 wherein the agent is selected from the group consisting of: sensitizers, dissolution inhibitors, photo-acid generators, biochemically active additives, thermally activated catalysts, light activated catalysts, electromagnetic radiation activated catalysts, hydration activated catalysts, pH activated catalysts, low melting agents, and enzyme activated catalysts.

18. The method according to claim 16 with the additional step of selectively introducing a catalyst that initiates a reaction or a series of reactions that results in an increased or decreased rate of erosion.

19. The method according to claim 18 wherein the reaction or series of reactions results in a decreased molecular weight of the polymer.

20. The method according to claim 18 wherein the reaction or series of reactions results in deprotection of the functional group.

21. A method of manufacture of an endoprosthesis comprising one or more alternate rates of erosion, said method comprising the steps:
providing a polymer comprising a relatively low rate of erosion;
embedding an agent for selectively increasing or decreasing the polymer's rate of erosion in the polymer;
fabricating an endoprosthesis from the polymer.

22. The method according to claim 21 wherein the agent is selected from the group consisting of: sensitizers, dissolution inhibitors, photo-acid generators, biochemically active additives, thermally activated catalysts, light activated catalysts, electromagnetic radiation activated catalysts, hydration activated catalysts, pH activated catalysts, low melting agents, and enzyme activated catalysts.

23. The method according to claim 21 with the additional step of selectively introducing a catalyst that initiates a reaction or a series of reactions that results in an increased or decreased rate of erosion.

24. The method according to claim 23 wherein the reaction or series of reactions results in a decreased molecular weight of the polymer.

25. A method of manufacture of an endoprosthesis comprising one or more alternate sets of mechanical properties, said method comprising the steps:
providing a polymer comprising a relatively low rate of erosion;
embedding an agent for selectively increasing or decreasing the polymer's rate of erosion in the polymer;
fabricating an endoprosthesis from the polymer.

26. The method according to claim 25 wherein the agent is selected from the group consisting of: sensitizers, dissolution inhibitors, photo-acid generators, biochemically active additives, thermally activated catalysts, light activated catalysts, electromagnetic radiation activated catalysts, hydration activated catalysts, pH activated catalysts, low melting agents, and enzyme activated catalysts.

27. The method according to claim 25 with the additional step of selectively introducing a catalyst that initiates a reaction or a series of reactions that results in an increased or decreased rate of erosion.

28. The method according to claim 27 wherein the reaction or series of reactions results in a decreased molecular weight of the polymer.

29. A method of treatment of a subject comprising the steps of:
providing an erodible endoprosthesis comprising one or more alternate rates of erosion;
implanting the endoprosthesis in the subject;
selectively initiating an increased or decreased rate of erosion of the endoprosthesis.

30. The method according to claim 29 wherein the increased or decreased rate of erosion continues for a relatively predetermined period of time.

31. The method according to claim 30 wherein the increased or decreased rate of erosion is followed by a relatively slower or faster rate of erosion.

32. The method according to claim 29 wherein the endoprosthesis is substantially completely eroded.

33. The method according to claim 29 wherein the step of selectively initiating an increased or decreased rate of erosion of the endoprosthesis comprises introducing a stimulus for initiation of a reaction or a series of reactions that result in an increased or decreased rate of erosion.

34. The method according to claim 29 wherein the endoprosthesis comprises an agent for selectively increasing or decreasing the rate of erosion of the endoprosthesis.

35. The method according to claim 34 wherein the step of selectively increasing or decreasing the rate of erosion of the endoprosthesis comprises introducing a stimulus to which the agent for selectively increasing or decreasing the rate of erosion is responsive.

36. The method according to claim 34 wherein the agent is selected from the group consisting of sensitizers, dissolution inhibitors, photo-acid generators, biochemically active additives, thermally activated catalysts, light activated catalysts, electromagnetic radiation activated catalysts, hydration activated catalysts, pH activated catalysts, low melting agents, and enzyme activated catalysts.

37. The method according to claim 33 wherein the stimulus is selected from the group consisting of: change in temperature, change in pH, light, electromagnetic radiation, hydration, one or more biochemical catalysts, and one or more enzymes.

38. The method according to claim 35 wherein the stimulus is selected from the group consisting of: change in temperature, change in pH, light, electromagnetic radiation, hydration, one or more biochemical catalysts, and one or more enzymes.

39. The endoprosthesis according to claim 6 wherein the endoprosthesis comprises an agent for initiating a reaction or a series of reactions that result in a transition from a first set of mechanical properties to a second set of mechanical properties.

40. The endoprosthesis according to claim 39 wherein the agent is selected from the group consisting of sensitizers, dissolution inhibitors, photo-acid generators, biochemically active additives, thermally activated catalysts, light activated catalysts, electromagnetic radiation activated catalysts, hydration activated catalysts, pH activated catalysts, low melting agents, and enzyme activated catalysts.

41. The endoprosthesis according to claim 39 wherein the agent is responsive to a stimulus.

42. The endoprosthesis according to claim 41 wherein the stimulus is selected from the group consisting of change in temperature, change in pH, light, electromagnetic radiation, hydration, one or more biochemical catalysts, and one or more enzymes.

43. The method according to claim 16 with the additional step of incorporating a therapeutic substance into the endoprosthesis.

44. The method according to claim 21 with the additional step of incorporating a therapeutic substance into the endoprosthesis.

45. The method according to claim 25 with the additional step of incorporating a therapeutic substance into the endoprosthesis.

46. The method according to claim 29 wherein the endoprosthesis further comprises a therapeutic substance.

47. The endoprosthesis according to claim 1 wherein said first rate and said second rate occur simultaneously.

48. The endoprosthesis according to claim 1 wherein said endoprosthesis further comprises an agent for terminating erosion.

49. The endoprosthesis according to claim 1 wherein said endoprosthesis is suitable for use in the treatment of strictures in lumens of the body, vascular disease, vascular disorders, cardiac rhythm disturbances, gastrointestinal disorders, ocular disease, ocular disorders, diseases of the spine, disorders of the spine, degeneration of bone, trauma to bone, muscular degeneration, trauma to muscle, to occlude obesity, urinary incontinence, or to occlude a lumen of the body.

50. The endoprosthesis according to claim 6 wherein said endoprosthesis is suitable for use in the treatment of strictures in lumens of the body, vascular disease, vascular disorders, cardiac rhythm disturbances, gastrointestinal disorders, ocular disease, ocular disorders, diseases of the spine, disorders of the spine, degeneration of bone, trauma to bone, muscular degeneration, trauma to muscle, to occlude obesity, urinary incontinence, or to occlude a lumen of the body.

51. The method according to claim 15 wherein said endoprosthesis is suitable for use in the treatment of strictures in lumens of the body, vascular disease, vascular disorders, cardiac rhythm disturbances, gastrointestinal disorders, ocular disease, ocular disorders, diseases of the spine, disorders of the spine, degeneration of bone, trauma to bone, muscular degeneration, trauma to muscle, to occlude obesity, urinary incontinence, or to occlude a lumen of the body.

52. The method according to claim 15 wherein said endoprosthesis is suitable for use in the treatment of strictures in lumens of the body, vascular disease, vascular disorders, cardiac rhythm disturbances, gastrointestinal disorders, ocular disease, ocular disorders, diseases of the spine, disorders of the spine, degeneration of bone, trauma to bone, muscular degeneration, trauma to muscle, to occlude obesity, urinary incontinence, or to occlude a lumen of the body.

53. The method according to claim 21 wherein said endoprosthesis is suitable for use in the treatment of strictures in lumens of the body, vascular disease, vascular disorders, cardiac rhythm disturbances, gastrointestinal disorders, ocular disease, ocular disorders, diseases of the spine, disorders of the spine, degeneration of bone, trauma to bone, muscular degeneration, trauma to muscle, to occlude obesity, urinary incontinence, or to occlude a lumen of the body.

54. The method according to claim 25 wherein said endoprosthesis is suitable for use in the treatment of strictures in lumens of the body, vascular disease, vascular disorders, cardiac rhythm disturbances, gastrointestinal disorders, ocular disease, ocular disorders, diseases of the spine, disorders of the spine, degeneration of bone, trauma to bone, muscular degeneration, trauma to muscle, to occlude obesity, urinary incontinence, or to occlude a lumen of the body.

55. The method according to claim 29 wherein said endoprosthesis is suitable for use in the treatment of strictures in lumens of the body, vascular disease, vascular disorders, cardiac rhythm disturbances, gastrointestinal disorders, ocular disease, ocular disorders, diseases of the spine, disorders of the spine, degeneration of bone, trauma to bone, muscular degeneration, trauma to muscle, to occlude obesity, urinary incontinence, or to occlude a lumen of the body.

56. The endoprosthesis according to claim 1 wherein said endoprosthesis comprises an endoprosthesis element comprising a plurality of apices alternating with a plurality of straight sections.

57. The endoprosthesis according to claim 56 wherein said endoprosthesis further comprises one or more connecting members.
